(19) United States
(12) Patent Application Publication
(10) Pub. No.: US 2004/0093093 A1
(43) Pub. Date: May 13, 2004

(54) Neural prosthesis

(75) Inventor: Brian J. Andrews, Edmonton (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: The Governors of the University of Alberta

(21) Appl. No.: 10/345,845

(22) Filed: Jan. 13, 2003

Related U.S. Application Data
(63) Continuation of application No. 08/810,820, filed on Mar. 5, 1997, now abandoned.

(30) Foreign Application Priority Data
Mar. 5, 1996 (CA) 2171067

(51) Int. Cl. A61F 2/70

(52) U.S. Cl. 623/25

(57) ABSTRACT

A neural prosthesis has a generator of electrical pulses, the pulses having a sine wave shape with frequency greater than 5 kHz, which may be amplitude modulated with a modulator, a blocking electrode for delivery of the electrical pulses to the neuron of the human nerve, the blocking electrode being electrically connected to the generator; and a controller operatively connected to the generator, the controller including an input for receiving control inputs, a control circuit responsive to the control inputs, and an output line responsive to the control circuit for sending output signals, the output signals of the controller including at least a start signal and a stop signal for controlling the generator. A method of controlling human nerve activity in a human body, the method comprising the step of applying electrical pulses to a neuron of a human nerve, the pulses being characterized by having a sine waveform and frequency over 5000 kHz such that, upon application of the pulses to a first site on the neuron, propagation of action potentials in the neuron is blocked at the first site. The neural prosthesis is used with a sensor having output representative of human body activity, such as body movement, muscle activity or nerve activity. For the prevention of an initial action potential, an initial pulse may be delivered with greater amplitude or different shape than subsequent pulses.

### FIELD OF THE INVENTION

This invention relates to neural prostheses.

### BACKGROUND OF THE INVENTION

A common requirement of many individuals with neurological disorders is the need to suppress unwanted and involuntary muscular contractions due to spasticity as well as stimulating contractions in paralyzed or weakened muscles. Clinically used nerve blocking techniques include injection of nerve or endplate blocking agents, antispasmodic medication or surgical procedures such as neurolysis, muscle section or lengthening and selective dorsal root rhizotomy. These techniques weaken muscle function temporarily or irreversible and can dramatically improve patients overall function.

In many cases the unwanted movements are stereotypical, phasic, triggered by voluntary motions often following primitive reflex patterns. In motor tasks such as locomotion, unwanted muscle action should ideally be dynamically suppressed before it can occur so that voluntary or FES induced movement can proceed unabated. In this way the affected muscle still retains its ability to contribute to controlled motion. For example: in many cases of spastic paralysis voluntary control is preserved to some degree but it is impaired by unwanted actions due to abnormally excessive activity in one or more muscle groups. This overactivity upsets the motion because the antagonist may not be able to overpower the unwanted opposition. Often the hyperactivity is in the more massive and stronger muscles. For example in the case of some hemiplegics due to stroke or cerebral palsy (type I, Gage J R (1990) Gait analysis in cerebral palsy, Clin. in Devel. Med. No. 121, Mac Keith Press, UK.), the main gait deficit is due to excessive plantarflexior activity as the knee is extended in late swing. As a consequence the toe contacts the floor rather than the heel resulting in an abnormal gait.

Apart from motion control there are other functional and therapeutic benefits to spasticity suppression. For example, excessive activity due to spasticity in young children or recent neurological impairment may be considered as a dynamic contracture i.e. the muscle can assume its normal length if this activity is blocked. If the muscle is not relaxed and allowed to be stretched for a sufficient periods it will lose sarcomers and become shorter and often ultimately leads to an irreversibly fixed contracture with consequence deformities that may require surgical intervention to correct.

The inventor has identified that, from the perspective of neuroprosthetic control, the ideal nerve blocking means should be reversible with no nerve damage. It should be selective with its action specific to predetermined groups of axons. It should be capable of rapid switching on and off to allow blanking of unwanted neuromuscular activity transients and duty cycle control. The degree of blocking should also be dynamically controllable by either selecting subsets of nerve axons for block or by changing the duty cycle of block in a given axon population.

While there have been some proposals of electrical nerve blocks in the prior art, these tend to have deficiencies. Existing suggestions for nerve blocks include:

DC block, often referred to as anodal block. Here a steady or slowly varying potential is applied to the nerve causing a reversible and selective local block. This technique has been used to demonstrate a natural recruitment order for FES (Petrofsky J S, Phillips CD, Impact of recruitment order on electrode design for neutral prosthetics of skeletal muscle, 1981 Am. J. Phys. Med. 60: 243-253.). The proportionality of DC block is questionable since axons show asynchronous activity when the block voltage is below a threshold (Campbell B, Woo M Y, Further studies on asynchronous firing and block of peripheral nerve conduction, 1966, Bull. of the Los Angeles Neurological Soc. 31(2): 63-71.).

Wedenski Block: Wedenski first described the phenomena in 1885. Here the nerve is stimulated at a high rate causing the rapid depletion of the neurotransmitter or calcium in the tubule system. This form of blocking has been proposed for neuroprosthetic control: normalizing recruitment order (see (a) McNeal D R., Bowman W W, Peripheral block of motor activity, In: Proc. Advances in External Control of Human Extremities, Ed. Garvilovic & Wilson, 1973, pp 473-519, Dubrovnik, ETAN Belgrade Yugoslavia; (b) Solomonow M., Eldred E, Lyman J., Foster J, Control of muscle contractile force through indirect high-frequency stimulation, 1983, Am. J. Phys. Med. 62(2): 71-82.; (c) Solomonow M, Eldred E, Foster J, Fatigue considerations of muscle contractile force during high-frequency stimulation, 1983, Am. J. Phys. Med., 62(3): 117-122; and (d) Solomonow M, King A, Shoji H, D'Ambrosia R, External Control of rate, recruitment, synergy and feedback in paralysed extremities, 1984, Orthopaedics, 7(7): 1161-1180.); spasticity suppression (Solomonow M, Shoji H, King A, D'Ambrosia R, Studies towards spasticity suppression with high frequency stimulation, 1984, Orthopaedics, 7(8): 1284-1288); bladder control (Ishigooka et al. 1994), The high frequency anti-dromic action potentials will collide with, and mutually annihilate, those generated by the cell body. Thus Wedenski block causes transmission blocking actions at all stages in the motor unit.

Collision Block: Here the nerve is stimulated by a spiral cuff electrode that generates unidirectional action potentials anti-dromically. Each anti-dromic pulse propagates towards the soma and will annihilate both itself and the first orthodromic action potential it meets. Any subsequent orthodromic will be annihilated at the site of the first collision until that point on the axon recovers from its refractory state. A complete block is obtained if the anti-dromic action potentials are repeated rapidly enough so that no naturally developed action potential can reach the electrode before an electrical pulse is generated. The maximal frequency for complete block is the reciprocal of the refractory period plus the transit time i.e. typically less than a few hundred hertz. This modality is being actively developed for human application (van den Honert C, Mortimer J T, Generation of unidirectionally propagated action potentials in a peripheral nerve by brief stimuli, 1979, Science, 26: 1311-1312; van den Honert C, Mortimer J T,. A technique for collision block of peripheral nerve: Frequency dependence, 1981, BME-28(5): 379-382; van den Honert C, Mortimer J T, A technique for collision block of peripheral nerve: single stimulus analysis, 1981, IEEE Trans. Biomed. Eng., BME-28(5): 373-378, Ungar I J, Mortimer J T, Sweeney J D, Generation of unidirectional propagation action potentials using a monopolar electrode cuff, 1986, Annals of Biomed, Eng., 14: 437-450.).

DC or galvanic block does not appear to have an important role in neuroprosthetics since in long term use will probably damage the nerve due to corrosive effects of the metal elctrode. The report of Campbell & Woo also questions its selectivity due to the asynchronous firing produced, with sub threshold voltage, in those fibers in-between those large diameter fibers that are truly blocked and those smaller fibers that remain unaffected.

Wedenski block is the only selective block since its effects are limited to those fibers stimulated. However, there appear to be potential drawbacks namely: the unavoidable powerful muscular contraction at the beginning of the blocking pulses until the neurotransmitter is sufficiently depleted to cause transmission failure. If the electrode generates anti-dromic pulses then these may cause painful sensations and unwanted reflex activity; nerve damage is associated with induced hyperactivity in the nerve (Agnew W F, McCreery D B, Neural Prostheses: Fundamental Studies, 1990, Prentice-Hall Inc. USA, pp 297-317.). If an epineurogram (ENG) detector were to be used the block would have to be first removed before the presence of spasticity could detected. Reestablishing the block would again induce a powerful muscle contraction. Also the use of sensory nerve ENG recording from distal electrodes is precluded. This modality is uniquely fiber diameter selective and allows proportional control of the block i.e. axons with decreasing diameters are blocked as the stimulus intensity is increased. However, duty cycle modulation of the block is not possible since time is required for the depleted neurotransmitter to be replenished before muscle contraction can begin and vice versa muscle contractions will continue until the transmitter is depleted at the block turn on.

Collision block appears to have some potential drawbacks: The intense stimulus will excite anti-dromic pulses not only in—motor neurons in a mixed peripheral nerve. This will also excite other pathways (posterior horn and Renshaw cells) that may cause discomfort or unwanted reflex activity. The surgical installation of a cuff will result in some handling of the nerve and may disrupt or constrict local blood supply at the time of installation and, if implanted into a child, may subsequently lead to nerve constriction as the child grows. The onset of the block is intuitively instantaneous, however, the turn-off time has not been reported. It will be at most twice the transit time plus any prolonged resetting of the cell body integrator due to the previous volley of anti-dromic input to various interneurons and dorsal column pathways.

### SUMMARY OF THE INVENTION

The inventor has proposed a new form of electrical nerve block for clinical use and the corresponding neural prosthesis in which the effects of the nerve block are local, that is the effects apply only at the site to which the block is applied and other parts of the nerve are not affected. In particular, undesirable continuous action potentials are not created, and therefore hyperactivity damage is avoided, and there are no unwanted reflex effects and it is painless.

There is therefore provided in accordance with one aspect of the invention, a neural prosthesis, comprising a generator of electrical pulses, the pulses being characterized by having a waveform such that, upon application of the pulses to an axon of a human nerve at a site on the axon, propagation of action potentials in the axon is blocked at the site, a blocking electrode for delivery of the electrical pulses to the axon of the human nerve, the blocking electrode being electrically connected to the generator; and a controller operatively connected to the generator, the controller including an input for receiving control inputs, a control circuit responsive to the control inputs, and an output line responsive to the control circuit for sending output signals, the output signals of the controller including at least a start signal and a stop signal for controlling the generator.

In accordance with a further aspect of the invention, there is provided a method of controlling human nerve activity in a human body, the method comprising the step of applying electrical pulses to an axon of a human nerve, the pulses being characterized by having a waveform such that, upon application of the pulses to a first site on the axon, propagation of action potentials in the axon is blocked at the first site.

Preferably, the neural prosthesis is used with a sensor having output representative of human body activity, such as body movement, muscle activity or nerve activity.

The waveform is preferably a sine wave with frequency greater than 5 kHz, which may be amplitude modulated with a modulator.

In a further aspect of the invention, a neural stimulator may be used to stimulate the same nerve to which the blocking generator applies electrical pulses.

For the prevention of an initial action potential, an initial pulse or pulse train may be delivered with asymmetric shape, or greater amplitude or different shape than subsequent pulses.

The proposed frequency range of the blocking pulses is similar to that proposed by Tanner in 1962 for experimental studies on frog nerves, and subsequently on frog and cat nerves by Campbell & Woo, (1964, Asynchronous firing and block of peripheral nerve conduction by 20 Kc alternating current, Bull. of the Los Angeles Neurological Soc., 29: 87-94, 1966, Further studies on asynchronous firing and block of peripheral nerve conduction, Bull. of the Los Angeles neurological Soc., 31(2): 63-71). Despite the long knowledge by some of this particular frequency, and its effect on frog and cat nerves, the waveform has not been positively proposed to be used for clinical applications to humans. Rattay 1990, Electrical Nerve Stimulation: Theory, Experiments and Applications, Springer Verlag, N.Y., mathematically models the use of a high frequency sine block at 2 kHz on a 10 μm unmyelinated nerve of the squid at 37° C., but uses an artificial excitation waveform at 500 Hz. This result cannot be extrapolated routinely to the clinical case at least in part since the blocking action may be affected by the harmonic relationship between the excitation frequency and the block frequency and in any event the block generates a single action potential.

These and further aspects of the invention are described in the description and claimed in the claims that follow.

### BRIEF DESCRIPTION OF THE DRAWINGS

There will now be described preferred embodiments of the invention, with reference to the drawings, by way of illustration, in which like numerals denote like elements and in which:

FIG. 1 is a schematic of a neural prosthesis according to an aspect of the invention;

FIG. 2 is a schematic of a neural prosthesis according to a second aspect of the invention;

FIG. 3 is a schematic of a neural prosthesis according to a third aspect of the invention;

FIG. 4 is a diagram showing an implanted electrode for use with the invention;

FIG. 5 is a graph showing pulse shape of blocking pulses in accordance with one aspect of the invention;

FIG. 6 is a schematic of a neural prosthesis according to a third aspect of the invention;

FIG. 7 is a set of traces showing the emg output of a child with spastic diplegia;

FIG. 8 shows the application of an embodiment of the invention to the leg of a patient;

FIG. 9 shows the application of a second embodiment of the invention to the leg of a patient; and

FIGS. 10A, 10B and 10C show respectively (A) a symmetrical square voltage waveform according to one aspect of the invention, (B) the equivalent current obtained during clinical application of the pulses of FIG. 10A to a human nerve, and (C) a prior art voltage waveform.

### DETAILED DESCRIPTION OF PREFERRED EMBODIMENTS

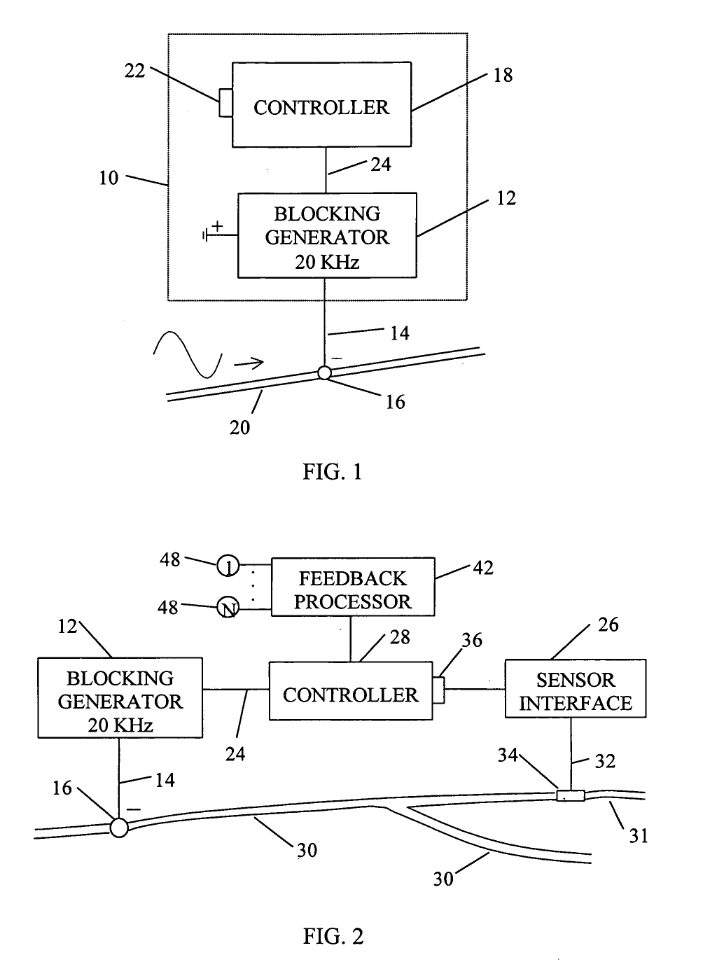

Basic elements of a portable neural prosthesis 10 are shown in FIG. 1, in which a generator 12 of electrical pulses is connected by conductor 14 to electrode 16. The generator 12 should be grounded in conventional manner, for example by grounding to the housing of the neural prosthesis 10. In operation, the electrode 16 is placed on or near a human nerve 20 for delivery of electrical pulses to an axon in the nerve 20. The electrode 16 may be a surface electrode, for application in the case of superficial nerves or an implantable electrode in the case of deep nerves. The generator 12 may for example be a conventional oscillator or a conventional programmable pulse generator. The generator 12 is controlled by a controller 18 having an input 22 and an output line 24. For implant use, it is preferred that the power supply for the neural prosthesis be a supercap or battery rechargeable inductively by an external coil.

In its simplest form, the control circuit of the controller 18 may be a manually operated momentary action on-off switch, in which a blocking signal is provided as long as a button is pressed, but more advantageously in many applications the input 22 may accept control input signals from one or more automated devices such as electronic sensors of human body activity and the control circuit may have any of various forms such as a rule induction circuit (as described in Andrews B J et al, 1989, Rule Based Control of a Hybrid FES Orthosis for Assisting Locomotion, Automedica, Vol. 11, p. 175-200, the content of which is herein incorporated by reference), a neural network (as described in Heller et al, Reconstructing muscle activation during normal working, Biol Cyber. 69:327:335 (1993), the content of which is herein incorporated by reference) an Adaptive Logic Network as described in Kostov et al, Machine Learning in Control of Functional Electrical Stimulation Systems for Locomotion, IEEE Trans. Biom. Eng. 42:6:541-551 (1995), the content of which is herein incorporated by reference) and using commercially available software such as ATREE Release 3.0 software, Dendronics Decisions Ltd. 1995, or using Rough Sets (as described in Andrews et al, Event Detection for FES Control Using Rough Nets & Accelerometers, Proc. 2nd Int. FES-Symp., 187-193, 1995, the content of which is herein incorporated by reference). While these control systems have previously been applied to nerve stimulation techniques, given the teaching in this patent document, they are readily adaptable to nerve blocking techniques. In the case of a simple manual switch, the output of the controller 18 consists only of a start signal and stop signal, either of which may be the presence or absence of current on the output conductor 14.

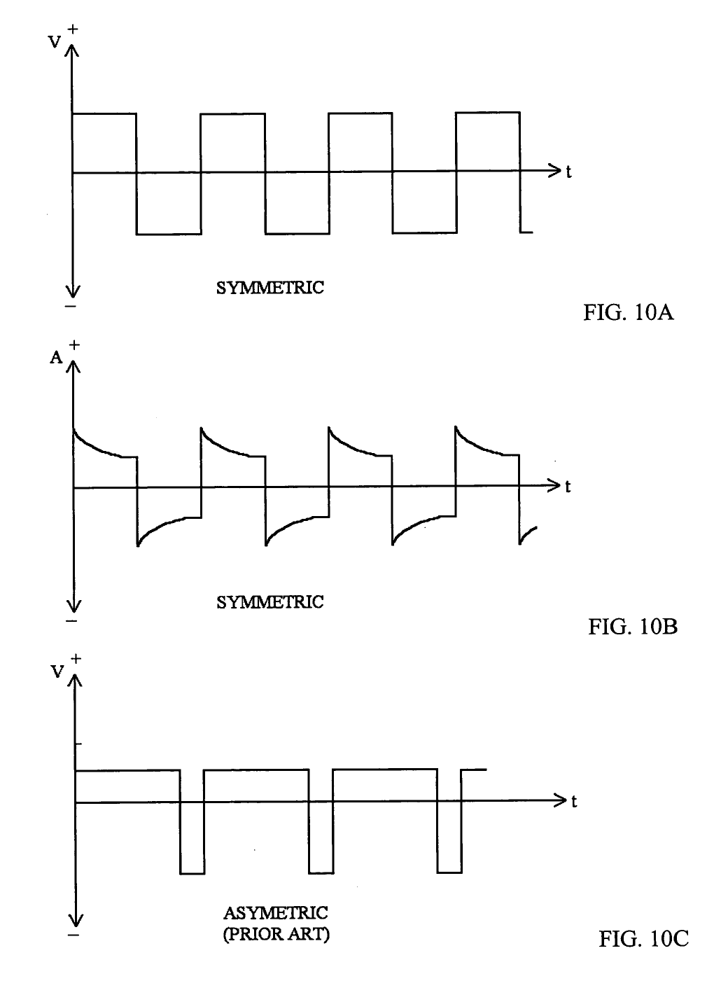

The electrical pulses generated by the generator 12 must be characterized by having a waveform such that, upon application of the pulses to an axon of a human nerve 20 at a site on the axon, propagation of action potentials in the axon is blocked only at the site. A waveform of a pulse is defined by its phase, amplitude and frequency. In this patent document, the amplitude of an electrical pulse will be discussed in terms of its voltage, but for each voltage there is a corresponding current produced at the electrode, and in some instances the amplitude may be discussed in terms of the current of the electrical pulse. Complicated shapes may be obtained that are the sum of many waveforms. An exemplary waveform is a sine wave having a frequency of greater than at least 5000 Hz. A blocking waveform of this type also has the additional benefit that it does not induce continuous action potentials in the nerve being blocked. For sine waves having frequencies between about 1000 Hz and 5000 Hz, some action potentials may propagate past the block site, although generally with increase of frequency and increasing intensity there is increased blocking. Generation of such a sine wave may commence with 0 voltage rising along a sine curve to a maximum of about 8 volts and then oscillating sinusoidally at, for example 20 kHz, between ±8 volts. The voltage depends on the distance to the nerve from the electrode, with greater voltage the further the electrode is from the nerve. At higher voltage, for example ±20 volts, a platinum electrode will begin breaking down. Thereafter the pulses are repeated until the block is no longer required. It is believed that in addition to a sine wave, symmetric waveforms will also work, for example, a square wave. For the square wave, the peak voltage may be slightly lower. A symmetric waveform is defined as having a positive current profile that is the mirror image, about the 0 current axis, of the negative current profile. An exemplary symmetric square waveform is shown in FIG. 10A. This shows the voltage applied to an electrode 16. The equivalent current produced at the electrode 16 is shown in FIG. 10B, showing the capacitative effect of the nerve membrane. An asymmetric profile is shown in FIG. 10C. The monophasic voltage spike 82 at 600 Hz, as reported in the prior art, is likely to be an excitatory input.

The symmetric waveform, however, will generate a single action potential in a human axon during onset of the block. To avoid this, the peak voltage of the pulses may be gradually increased, but this delays the onset of the block. Preferably, an initial pulse or pulse train is generated, upon receipt by the generator 12 of a start signal, that has greater amplitude than subsequent pulses, as for example shown in FIG. 5, for example at least twice the amplitude of subsequent pulses. In this case, the initial action potential induced by the onset of the block is eliminated. This initial pulse may also have a different shape (for example, square) than subsequent pulses, or the initial pulses may be asymmetric, with subsequent pulses symmetric as shown for the pulses in FIG. 5. The first two pulses of FIG. 5 are asymmetric, with the remainder symmetric. Overall, through the period during which the pulses are applied to a nerve, the charge delivered by the electrode should be balanced to avoid electrode galvanic corrosion and damage to the nerve.

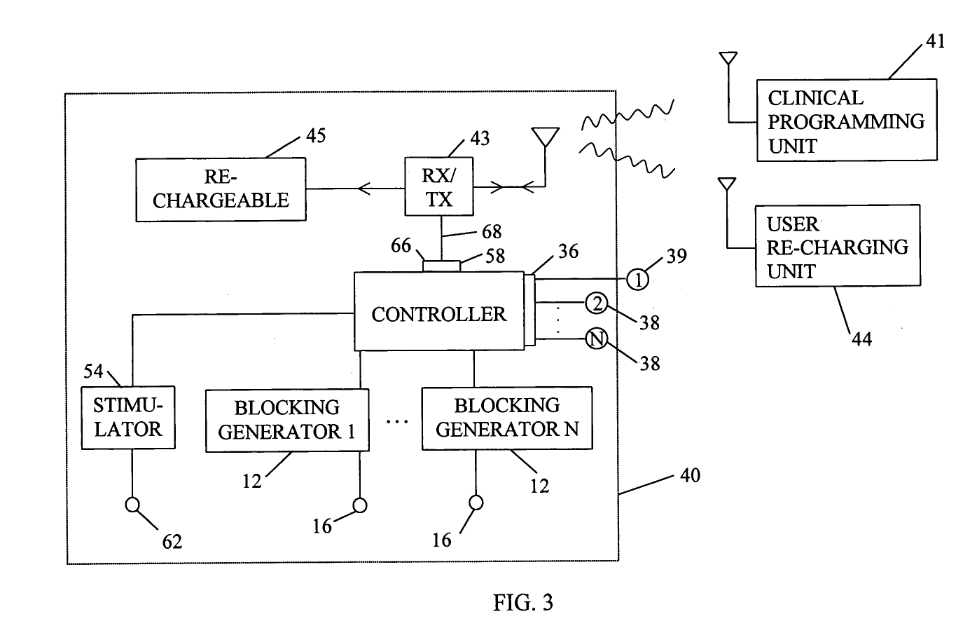

A configuration of neural prosthesis suitable for implants is shown in FIG. 3. The implantable neural prosthesis 40 includes controller 58, which receives inputs from sensors 38 contained within the neural prosthesis 40 and from sensors 39 outside the neural prosthesis 40. The neural prosthesis 40 is remotely controlled by a clinical programming unit 41 that communicates with a transceiver 43 contained within and housed with the implantable neural prosthesis 40. Controller 58 may be a digital signal processor or general purpose computer programmed in accordance with the principles set out in this patent document. For example, machine learning, if used, may be carried out in the controller 58.

Power signals are transmitted by user re-charging unit 44 to the transceiver 40, and stored in re-chargeable power unit 45. The re-chargeable power unit 45 may be a high capacity capacitor or rechargeable battery. It is preferred that the re-chargeable power unit not be of some NiCad types, since some NiCad batteries produce gas and are not suitable for implants. On the other hand, for stroke patients whose cognitive function may be impaired, it may be desirable to locate the re-charging unit 44 in a bed or chair or other object which the patient frequently uses so as to reliably re-charge the re-chargeable power unit 45. The user re-charging unit 44, re-chargeable power unit 44 and transceiver 43 are each available in the art in themselves, while the clinical programming unit 41 is a general purpose computer with transceiver attached that may be readily programmed to carry out the procedures described in this patent document.

Control signals are provided along line 68 to input 66 of the controller 58. The controller 58 may interrogate the sensors 38, 39 and send stop and start signals to blocking generators 12 and stimulator 54. If desired, the voltage supplied to the electrodes 16 may be amplitude modulated to control the size of nerve blocked by the electrical pulses. Control signals for this purpose may be sent from the clinical programming unit 41, which typically may include a computer, additional sensors and patient operated switches. For example, patient operated switches may be used in walking during supervised learning to indicate when a given movement is desired. The computer may then correlate the intended movement with the input of the sensors to provide an alternative to the patient operated switch.

The clinical programming unit 41 may be used to train for example a self-adaptive learning algorithm in the controller 58 by giving it known examples to begin the learning process. The clinical programming unit 41 may be used in addition to change stimulus or blocking intensity or duration of blocking or stimulus of an implant.

As illustrated in FIGS. 2 and 3, a controller 28 or 58 may receive control inputs at input 36 from one or more sensors 26, 38 and 39 of human body activity. The sensor 26 may be a conventional electroneurogram connected to a sensor branch 31 of nerve 30 or connected directly to the nerve 30 through conductor 32 and cuff 34. The nerve to which the sensor 26 is attached may also be in a different part of the body from the blocking generator 12 with which it is used. In this instance, the sensor 26 generates a signal indicative of human nerve activity which is used as an input to controller 28. The sensors 39 may also be sensors of neural activity or may be sensors of human body movement, including muscle contraction, human body activity preparatory to a given movement. Such sensors are known in the art in themselves.

Examples of sensors used in the open loop condition of the control circuits exemplified by FIGS. 1, 2 and 3 include (a) electromechanical transducers such as push-button switches, finger pressure or force sensors, rate gyroscopes joint angle displacement, velocity or acceleration sensors, inclinometers and potentiometers, (b) voice or sound input through a microphone and (c) electrodes sensing electrical or magnetic biophysical events such as brain signals (EEG), nerve signals, electrical or sonic muscle signals.

In the closed loop condition, also illustrated in FIGS. 2 and 3, in which a feedback processor 42 receives signals from sensors 48, exciting or blocking stimuli are sensed by the sensors 48 and used as feedback or feed-forward to the controller 28 form subsequent outputs for control of the generator 12. Examples of sensors used in the closed loop condition include: (a) strain gauge transducers or pressure sensors that sense force actions, such as in braces shoes or other structures attached to the patient and crutches, sticks, walking frames or other forms of walking aid, (b) accelerometers attached to a patient or walking aid, (c) gyroscopes attached to the patient or walking aid, (d) position sensors attached to limb segments or mechanically encompassing anatomical joints that sense the relative linear motion or angulation of limb segment such as electromagnetic transmitters/receivers, magnetic field sensors, ultrasonic transmitter/receivers, fiber optic motion switches or goniometers, resistive, potentiometric, electromagnetic or optical goniometers and (e) natural sensors monitored through electrodes sensing brain, nerve or muscle action potentials.

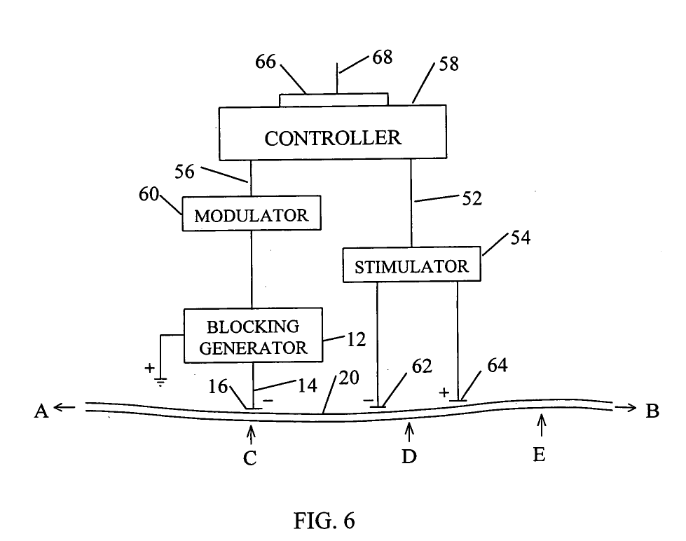

The neural prosthesis thus described may be used to add additional outputs to existing FES systems, for example painless selective nerve block, and bidirectional or unidirectional nerve stimulation. An application is illustrated in FIG. 6.

Controller 58 is attached via lead 52 to a conventional stimulator 54, and via output 56 to modulator 60 attached to blocking generator 12. Blocking generator 12 is connected by lead 14 to an electrode 16 located in conduction contact on or over or around a site C on the nerve 20. On the same nerve, but at an adjacent site D, the stimulator 54 is likewise in conduction contact with the nerve via electrodes 62 and 64, which may be for nerve cuff electrodes. At a signal from controller 58, which may be a microprocessor programmed with any of several conventional control techniques for stimulation of nerves, the stimulator 54 applies electrical stimulation pulses to the nerve 20. Such pulses may be a trapezoidal waveform. At the same time, or at least before an action potential can propagate from the electrode 62 past site C, blocking generator 12 is turned on by a signal from the controller 58 to effect a block of any action potentials stimulated in nerve 20 and propagating in direction A.

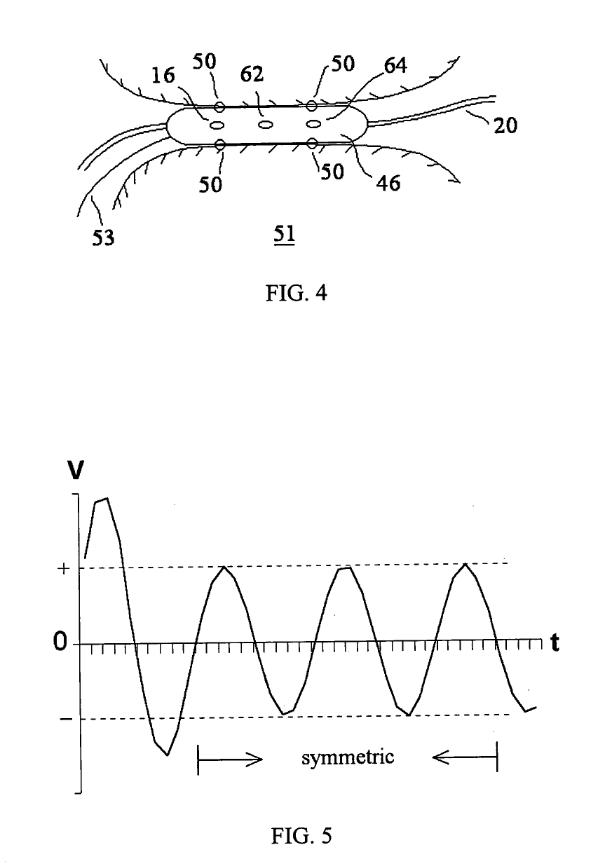

The electrodes 62 and 64 may form half of an asymmetric tripolar cuff described in Fang & Mortimer, Selective activation of small motor axons by quasitrapezoidal current pulses, IEEE Trans. Biomed. Eng., 38:2, 168-174, but it may also be another stimulus. An implanted version of the electrodes 16, 62 and 64 is shown in FIG. 4. Cuff 46 is sutured at 50 to the body 51 around a nerve 20. Pulses are applied through cable 53. In this instance, cathode 62 excites all fibers in the nerve 20 and anode 64 selectively blocks the orthodromicly propagating potentials according to their diameter and the controllable DC current applied to the electrodes. This provides natural firing order of motor neurons, and use of the blocking electrode at site C blocks unwanted anti-dromicly propagating action potentials.

Thus, in the case where nerve 20 is a mixed nerve including afferent neurons, and direction A is anti-dromic (in the direction of the soma) then motor neuron stimulation may be induced orthodromicly (direction B) without unwanted antidromic action potentials propagating in the nerve, and hence without unwanted painful side effects.

In the case where direction A is orthodromic, and orthodromicly propagating action potentials are generated at site D, the controller 58 may be programmed to instruct modulator 60 to modulate the electrical pulses by gradually decreasing the voltage of the pulses applied by the blocking generator 12 from a supramaximal level while a. stimulus is applied to nerve 20 at site D. This will have the effect of causing a block for all nerves initially and then sequentially unblocking larger and larger neurons as the voltage of the blocking pulses is decreased. Therefore, when it is desired to stimulate motor nerves in the natural order (order of increasing size), without stimulating smaller diameter afferents, and the stimulus stimulates motor nerves in order of decreasing size (reverse order) the blocking effect may be used sequentially with the stimulator applying stimulation to the motor neurons to create a natural firing order of the motor neurons. That is, at supramaximal stimulus, all motor neurons will be firing in nerve 20. The amplitude of the blocking pulses should initially be supramaximal: all motor neurons will be blocked locally and without generating any action potentials themselves. As the amplitude of the blocking pulses is decreased, smaller motor neurons may be selectively unblocked resulting in stimulated action potentials propagating in direction A in smaller nerves.

In general, two blocking electrodes may be placed on either side of a stimulating electrode, with a complete block on one side of the stimulating electrode and a selective block on the other side. The amplitude of the excitatory stimulus and the amplitude of the partial block may select any band of fibers in the nerve based on fiber diameter for unidirectional stimulus in either the antidromic or orthodromic direction.

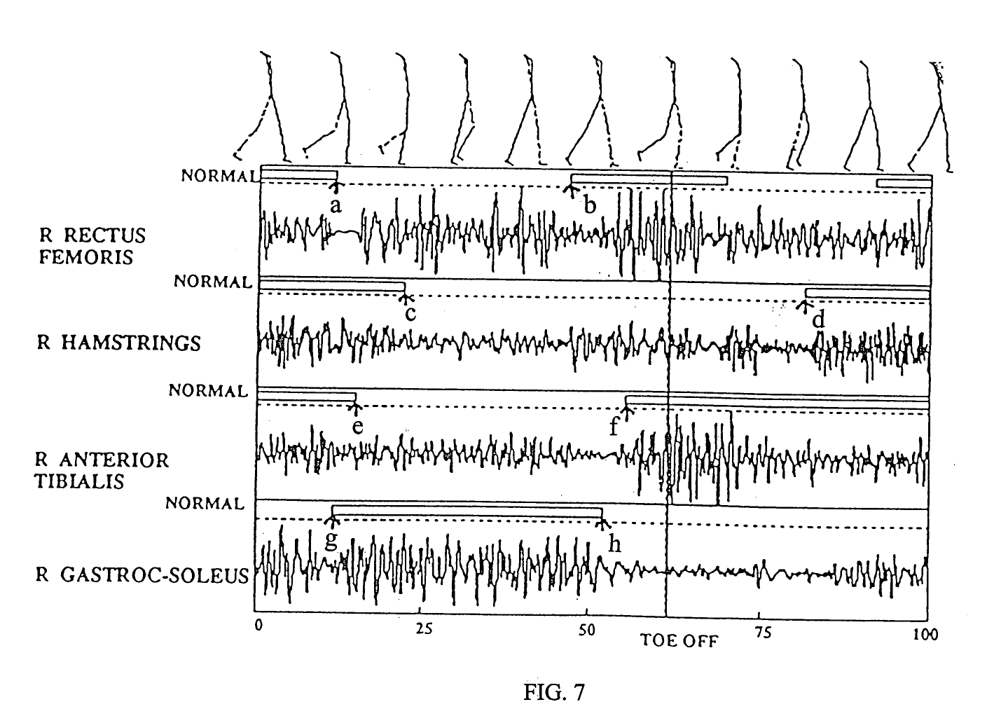

A typical application includes correction of the gait of a neurologically impaired patient. FIG. 7 shows the periods during the gait cycle in which inappropriate muscle activity is observed. The role of the neural prosthesis is to block neural activity in the periods indicated in FIG. 7. To delineate the desired start and stop blocking, the eight events for each leg (labelled as events a-h in the figure) need to be detected in real time as the gait proceeds. The neural prosthesis outputs a binary decision (on-off) to each blocking generator 12 located on neurons leading to the indicated muscles. These are: femoral nerve for rectus femoris, sciatic nerve for the hamstrings, common peroneal nerve for the anterior tibialis and tibial nerve for the gastroc-soleus. In this example, the block is a two state on or off applied either maximally blocking all traffic in the nerves or not. Thus, the block to, femoral nerve, innervating the rectus femoris, would start at point a and be maintained until point b. In the same way the motor nerve branches of the sciatic nerve would be blocked during the period c to d. The common peroneal nerve is blocked in the period e to f, and the tibial nerve from h to g.

In this instance, it is preferred that human body activity preparatory to a given human body movement is sensed, such as a foot plant or weight shift, by any of various sensors, and body movement is predicted based on the information received from the sensors. The electrical pulses are then applied to a nerve, such as the tibial nerve, used in the human body movement.

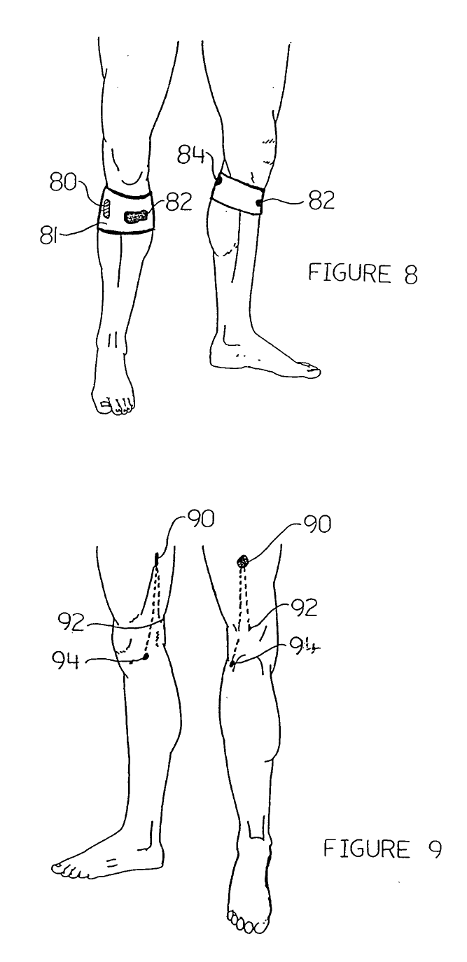

In a further example, control of the hemiplegic ankle joint may be obtained. In some neurologically impaired patients, for example the type 1 cerebral palsy child, the foot may drop during a leg swing and prematurely contact the ground. The problem manifests itself during late swing. As the knee is extended, the ankle plantar flexors contract, thus bringing the front of the foot down. To solve this problem, as shown in FIG. 8, neural prosthesis using sensor 80 is attached with an elastic band 81 to the leg with a common electrode 82, and a blocking surface or percutaneous electrode 84 over the tibial nerve. The sensor 80 senses the location of the leg during the swing by detection of muscle signals corresponding to the swing of the leg, although the system may also use a sensor of human body position, for example the actual movement of the leg. Upon occurrence of a signal *from the sensor, a controller 28 of the neural prosthesis instructs a blocking generator 12 (not shown in FIG. 8) to apply electrical pulses to the blocking electrode 84. Thus, as the leg swings forward, the ankle flexors are blocked and the swing is normal. Alternatively, as shown in FIG. 9, an implanted neural prosthesis 90 may be used, with implanted blocking electrode 92 on the tibial nerve and a stimulating electrode 94 on the common peroneal nerve. The stimulus is a standard stimulus to contract the tibialis anterior and lift the foot during swing.

In addition, during the swing phase of a neurologically impaired patient, the knee extensor sometimes inappropriately contracts. In this instance, the block may be applied to the femoral nerve during the swing phase.

For the tibial nerve, surface electrodes may be used. However, for deeper nerves there is a risk that a current density high enough to effect a block will burn the skin. Hence, the surface electrodes can only be used on superficial nerves.

The modulator 60 may be used to increase or decrease the amplitude of the electrical pulses output by the blocking generator 12. The increase/decrease may also be repeated. As for example, it often occurs in the stroke patient that unwanted neural activity in the arm neurons, for example the median nerve, cause the arm flexors to contract and cause the arm to be held tightly against the body, with the fist clenched. By detecting activation of the arm extensors, a variable block can be selectively and repetitively applied to the arm flexors to allow the arm to gradually flex. In some stroke patients, unwanted neural activity in the nerves of the arm causes both the flexors and extensors to tighten. Since the flexors are stronger than the extensors, the arm is pulled inward to the body and the fist clenched. Application of electrical pulses to cause local blocking of motor neurons for the flexors, thus may be used to allow selective arm movement.

In a further example of the method of operation of the neural prosthesis as illustrated in FIG. 6, the blocking electrodes are placed in conduction contact with a branch of the pudendal nerve that controls the bladder. One or more sensors 38, for example of nerve signals, muscular activity or movement, signal to a controller 28 when the bladder contracts, and the controller 28 instructs one of the blocking generators 12 to locally block the pudendal nerve, and thus prevent contraction of the sphincters in the urinary tract. In some cases, a unidirectional stimulus to the anterior sacral roots (S₂ and S₃) of the spinal chord, as for example using the neural prosthesis configuration shown in FIG. 3 with stimulator 54, may then be used to stimulate both the bladder (detrusor) and the sphincter. As the bladder contracts under the stimulus or naturally, stimulus of the sphincter is blocked and an approximation of normal function may be obtained. In this instance, the application of the stimulus and the block may be initiated directly using input from the patient to the controller at 66. The input 66 may be for example a direct mechanical input (push button) or indirect, using a sensor of some activity by the patient connected via line 68. Reflexive activity often prevents the bladder from filling properly in between voiding. Presently, the posterior spinal roots are cut. Use of the blocking technique of the present invention to block the posterior sacral roots is believed to be a preferable treatment.

In a further application of the neural prosthesis, the configuration of FIG. 3 in combination with the configuration of FIG. 1, may be applied to restore male sexual or reproductive function. Stimulator 54 applies a low frequency 9 Hz stimulation to the S₂ nerve root at site D. This frequency should be low enough that bladder and bowel function is not stimulated. Blocking generator 12 is applied to site C, in the orthodromic direction A, with its blocking amplitude adjusted to block nerve fibers with larger diameter fibers. At a third site E, more proximal to the spinal chord than site D, hence in the antidromic direction B, a complete block is applied to the S₂ root using a blocking waveform generated for example by the blocking generator 12 of FIG. 1, or a further blocking generator 12 controlled directly by controller 58. In this instance, the controller 28 only need be a manually operated switch for example a magnetic reed switch that may be operated by bringing a magnet close to the skin.

In a further application of the neural prosthesis, the hypogastric plexus where it lies in front of the left common iliac vein may be stimulated to effect electroejaculation while a blocking generator 12, for example using the configuration of FIG. 3, may be used to apply AC blocking electrical pulses to a site C more proximal to the spinal chord than site D. In this instance, antidromic neural activity (in the direction A) generated by the stimulator 54 is blocked.

In a further application, it is believed that occlusive sleep apnea (OSA) may be reduced by applying a unidirectional orthodromic stimulus to the medial pterygoid nerve using the neural prosthesis of FIGS. 3 or 6. Antidromic activity (direction A) would be blocked by a blocking generator. Since the nerve is deep, an implant system is required. The stimulator 54 may be switched on and off by the use of an accelerometer with dc response that would sense when the head was at the appropriate inclination for OSA. Alternatively, the sensor 38 may be a magnetic field sensor sensing the earth's magnetic field, an inclinometer or a tilt switch or a combination of such sensors.

There are some surgical considerations regarding electrodes and thus the mode of block. Generally the spiral self wrapping nerve cuff electrodes used for collision block (Agnew W F, McCreery D B, 1990) appear to be safe provided they are sufficiently slack.. Stein et al. 1977, (Stable long-term recordings from cat peripheral nerves), Brain Res, 128: 21.) observed some loss of larger-diameter myelinated axons with implanted peripheral nerve cuffs less than 40% greater in diameter than the nerve. However if these devices are used in children they must retain at least this degree of slackness throughout growth e.g. Peacock et al. 1987, (Cerebral palsy spasticity: Selective dorsal rhizotomy, Pediatric Neuroscience, 13, 61-66.) advocates selective, partial dorsal root rhizotomy to spastic muscle tone in the cerebral palsied child and that the procedure be carried out when the child is about 4 or five years old, before the dynamic muscle contractures become fixed. One may expect a small change in nerve diameter during maturation and, although cuff electrodes may be installed with slack, they will quickly be infiltrated with fibrous tissue and the combination may over time become constrictive. Cuff electrodes, particularly of the tripolar type, have the advantage of reducing the current required to block and making the blocking effect more uniform over the cross-section of the nerve.

Monopolar electrodes do not appear to have the same concerns, but do not have all the advantages of cuff electrodes, and therefore are believed to be equally preferable to cuff electrodes. For example, a conventional 2.5 mm platinum iridium button may be used with a silastic skirt to allow suture to adjacent tissue thus forming a tissue channel in which the nerve is free to move. These electrodes have been used successfully since 1991 for electrical stimulation of nerves to restore functional movements to a paraplegic.

Using a nerve model based on voltage clamp experimental data based on rat nodes (which closely represents human nerve), the inventor has observed blocking over a range of frequencies from 5-20 kHz. The blocking mechanism appears to depend on the response of the voltage gated ion channels of the neuron to the blocking action, and specifically appears to result from blocking of the sodium channels of the neuron. The node where the blocking potential is applied cannot stay in a depolarized state long enough to conduct a propagating action potential to the next node. This appears to be the case for any phase difference between the stimulus, potential and the blocking signal.

A person skilled in the art could make immaterial modifications to the invention described in this patent document without departing from the essence of the invention that is intended to be covered by the scope of the claims that follow.

The embodiments of the invention in which an exclusive property or privilege is claimed are defined as follows:

1. A neural prosthesis, comprising:
a generator of electrical pulses, the pulses being characterized by having a waveform such that, upon application of the pulses to an axon of a human nerve at a site on the axon, propagation of action potentials in the axon is blocked only at the site;
a blocking electrode for delivery of the electrical pulses to the axon of the human nerve, the blocking electrode being electrically connected to the generator; and
a controller operatively connected to the generator, the controller including an input for receiving control inputs, a control circuit responsive to the control inputs, and an output line responsive to the control circuit for sending output signals, the output signals of the controller including at least a start signal and a stop signal for controlling the generator.

2. The neural prosthesis of claim 1 further including a sensor having output representative of human body activity, the sensor being connected to the input of the controller.

3. The neural prosthesis of claim 1 in which the electrical pulses are characterized by having a symmetric waveform.

4. The neural prosthesis of claim 3 in which the electrical pulses are characterized by having a frequency greater than about 5 kHz.

5. The neural prosthesis of claim 1 further including a modulator operatively connected to the generator for amplitude modulating the electrical pulses.

6. The neural prosthesis of claim 2 in which the sensor is a sensor of human nerve activity in a pre-determined nerve and the electrical impulses are characterized by having a waveform such that, upon application of the pulses to the pre-determined nerve, propagation of action potentials in the pre-determined nerve is blocked.

7. The neural prosthesis of claim 6 further including:
a neural stimulator operatively connected to the controller; and
stimulation electrodes electrically connected to the neural stimulator.

8. The neural prosthesis of claim 1 further including:
a neural stimulator operatively connected to the controller; and
stimulation electrodes electrically connected to the neural stimulator, whereby a unidirectional nerve stimulator is formed.

9. The neural prosthesis of claim 1 in which the electrodes are surface electrodes.

10. The neural prosthesis of claim 1 in which the generator includes a circuit for delivering to the blocking electrode an initial pulse with greater amplitude than subsequent pulses.

11. The neural prosthesis of claim 1 in which the generator includes a circuit for delivering an initial pulse having a different shape than subsequent pulses.

12. The neural prosthesis of claim 1 further including:
a first transceiver housed with the controller;
a remote programming unit; and
a second transceiver operatively connected to the remote programming unit.

13. The neural prosthesis of claim 1 further including:
a first transceiver housed with the controller;
a remote re-charging unit; and
a remotely chargeable power supply housed with the controller.

14. The neural prosthesis of claim 3 in which the electrical pulses have a symmetric shape.

15. A method of controlling human nerve activity in a human body, the method comprising the steps of:
applying electrical pulses to a neuron of a human nerve, the pulses being characterized by having a waveform such that, upon application of the pulses to a first site on the neuron, propagation of action potentials in the neuron is blocked only at the first site.

16. The method of claim 15 further including the step of:
applying the electrical pulses to a neuron of a human nerve upon sensing neural activity in the neuron.

17. The method of claim 16 in which the human nerve is an afferent nerve.

18. The method of claim 17 in which the electrical pulses are applied through surface electrodes.

19. The method of claim 15 further including the step of:
applying the electrical pulses to a neuron of a human nerve upon sensing of a pre-determined body movement of the human body.

20. The method of claim 19 in which:
the pre-determined body movement is contraction of the bladder; and
the neuron to which the electrical pulses are applied is in a branch of the pudendal nerve that controls the sphincter.

21. The method of claim 20 further including:
applying a unidirectional electrical stimulus to the sacral roots to stimulate the bladder to contract.

22. The method of claim 19 in which:
the pre-determined body movement is a swinging of a foot forward; and
the neuron to which the electrical pulses are applied is a motor neuron in the tibial nerve.

23. The method of claim 19 further including:
sensing human body activity preparatory to a given human body movement; and
applying the electrical pulses to a nerve used in the human body movement.

24. The method of claim 15 further comprising:
applying the electrical pulses to a neuron through human skin using a surface electrode.

25. The method of claim 15 further including modulating the electrical-pulses.

26. The method of claim 25 in which modulating the electrical pulses includes ramping the amplitude of the electrical pulses.

27. The method of claim 15 further including:
applying an electrical stimulus to the human nerve at a second site on the same human nerve.

28. The method of claim 26 in which the first site is adjacent the second site.

29. The method of claim 27 further including:
modulating the electrical pulses.

30. The method of claim 15 further including commencing application of the electrical pulses with a first electrical pulse whose amplitude is greater than the amplitude of subsequent electrical pulses.

31. The method of claim 15 in which the nerve to which the electrical pulses is the pudendal nerve.
